(19) United States
(12) Patent Application Publication
(10) Pub. No.: US 2022/0250397 A1
(43) Pub. Date: Aug. 11, 2022

(54) Method For Ink-Jet Printing A Silicone-Based Composition Onto A Textile Substrate

(71) Applicant: Sigvaris AG, St. Gallen (CH)

(72) Inventors: Guillaume BOUTTE, SAINTE-FOY-L'ARGENTIERE (FR); Dorian FAYOLLE, VIRICELLES (FR)

(73) Assignee: Sigvaris AG, St. Gallen (CH)

(21) Appl. No.: 17/617,440

(22) PCT Filed: Jun. 11, 2020
(86) PCT No.: PCT/EP2020/066237

§ 371 Date: Dec. 8, 2021

(30) Foreign Application Priority Data
Jun. 11, 2019 (FR) FR1906202

(51) Int. Cl. B41J 3/407; B41M 5/00; D06M 15/643; A41B 11/12; B41J 2/145; D06B 11/00; D06M 23/16; A61F 13/08; D06P 5/30; D06P 1/52; D06P 1/54; C09D 11/30; C09D 11/102

(52) U.S. Cl. CPC B41J 3/4078; D10B 2501/021; D06M 15/643; A41B 11/128; B41J 2/145; D06B 11/0059; D06B 11/0073; D06M 23/16; A61F 13/08; D06P 5/30; D06P 1/5292; D06P 1/54; C09D 11/30; C09D 11/102; B41M 5/0023

(57) ABSTRACT

A method for ink-jet printing a two-component silicone-based composition onto a textile substrate is presented. The composition includes a first component having at least one silicone polymer and a second component having at least one cross-linking agent. The method includes the following steps: providing a printing system having at least one print head, itself having at least one print nozzle of spraying the two-component silicone-based composition onto the textile substrate, and a device for producing relative movement between the print head and the textile substrate, spraying the first component and the second component onto the textile substrate via the print nozzle cross-linking the silicone polymer by reaction between the first component and the second component so as to form a printed pattern on the textile substrate.

### TECHNICAL FIELD

The present invention relates to a method for inkjet printing a silicone-based composition onto a textile substrate, and to an inkjet printing system for carrying out such a method.

The field of the invention is that of the manufacture of textile articles comprising one or more zones coated with a material comprising one or more silicone polymers, the material preferably being intended to come into contact with the skin.

By way of non-limiting examples, the invention makes it possible to manufacture textile articles of the compression and/or support device type, medical compression orthosis type, town everyday stocking type, sports pantyhose type (compression or not), comfort compression pantyhose type, etc.

### PRIOR ART

To apply a composition to a textile substrate, a conventional method is coating. Coating consists of covering the textile substrate with a layer of polymer, in particular a layer of polyvinyl chloride (PVC), polyurethane (PU), or in fact silicone, for example.

When silicone is used, the textile substrate is coated with a curable silicone composition in order to form solid silicone coatings. The silicone composition conventionally comprises a silicone polymer, for example polydimethylsiloxane (PDMS), a cross-linking agent, and optionally a catalyst. When the composition is applied to the surface of the textile substrate, cross-linking of the silicone polymer occurs (polyaddition or polycondensation), resulting in the formation of a cross-linked silicone material.

The silicone composition may be of the one-component or two-component type. In the case of a two-component silicone composition, the latter typically comprises a first component comprising a silicone polymer, and a second component comprising a cross-linking agent which makes it possible to initiate cross-linking of the polymer, and optionally a catalyst. The first component may also be termed a resin. Cross-linking is only possible when the two components are mixed together. In the case of a one-component silicone composition, the silicone polymer and the cross-linking agent are contained in a single component, and the cross-linking is initiated when said component is sprayed onto the textile substrate, by contact with air or by heating to a high temperature.

However, the coating method suffers from disadvantages.

One of these disadvantages is that this method is not suitable for producing precise and relatively complex patterns produced on predetermined zones of the textile substrate. In fact, the zones of the textile substrate covered with the silicone composition are generally wide and coarse, and are typically in the form of bands. The production of patterns such as geometric figures of the circular, round or square type, for example, or indeed of more complex patterns or representations is limited with this method, in particular the coating of shapes or dots of the order of a micron.

Another method exists for applying a silicone-based composition to a textile substrate. This is inkjet printing. The expression “inkjet printing” is generic, insofar as the printing is not limited to an ink per se, but can also be used to print other materials such as silicone, for example.

As is the case with inkjet printing, the silicone-based composition is sprayed onto the textile substrate in the form of droplets.

Examples of systems for printing silicone onto a textile substrate are known from documents CH 678867 and KR 20060094607.

The systems described in those documents comprise a print head provided with a single row of nozzles, which limits the printing speed and therefore the productivity. In fact, a single row of nozzles imposes a slower speed of travel of the textile in order to allow optimized coverage of the material. In order to increase productivity, accelerating the spraying speed of the composition would require the print nozzles to be subjected to greater stresses, accelerating their wear.

### DESCRIPTION OF THE INVENTION

The objective of the present invention is to overcome the disadvantages of the prior art.

In particular, the aim of the invention is to provide a method for inkjet printing a two-component silicone-based composition.

The invention also aims to provide a printing method of this type, making it possible to increase the rate of production of the printed textiles, in particular in order to obtain a rate of production which is similar to or at least close to that for coating.

To this end, the invention concerns a method for inkjet printing a two-component silicone-based composition onto a textile substrate, said composition comprising a first component comprising at least one silicone polymer and a second component comprising at least one cross-linking agent, the method comprising the following steps:
providing a printing system comprising at least one print head itself comprising at least one print nozzle which is capable of spraying the two-component silicone-based composition onto the textile substrate, and means for relative displacement between the print head and the textile substrate,
spraying the first component and the second component onto the textile substrate via said print nozzle,
cross-linking the silicone polymer by reaction between the first component and the second component in order to form a pattern printed on the textile substrate.

The method in accordance with the invention makes it possible to print a pattern onto a textile substrate, this pattern comprising a cross-linked silicone polymer derived from a two-component composition.

In accordance with other advantageous characterizing features of the invention, taken individually or in combination:
cross-linking of the silicone polymer is initiated by mixing the first component and the second component on the textile substrate or before coming into contact with the textile substrate;
the first component is preferably the major component in the composition, i.e. the component with the highest content by weight with respect to the total weight of the composition;
the first component preferably comprises at least 50% by weight of silicone polymer with respect to the total weight of the first component, preferably at least 70% by weight of the silicone polymer with respect to the total weight of the first component. A high silicone content makes it possible to obtain a printed material predominantly composed of a cross-linked silicone polymer;
the silicone polymer is compatible with the skin. The term “compatible” means that the silicone polymer does not present any biological or toxicological risks when in contact with the skin and does not cause a skin reaction;
the printing system comprises at least one first print nozzle which is capable of spraying the first component, and at least one second print nozzle which is capable of spraying the second component;
the first print nozzle and the second print nozzle belong to the same print head. This makes it possible to improve the rate of production of the textile covered with silicone material (what is known as the printed textile), while preventing the silicone polymer from cross-linking in the print nozzles, or at least in the printing system;
the first print nozzle and the second print nozzle belong to two distinct print heads. The first print nozzle belongs to a first print head, and the second print nozzle belongs to a second print head;
at least one print nozzle comprises two outlet orifices, including a first outlet orifice which is capable of spraying the first component, and a second outlet orifice which is capable of spraying the second component;
at least one print head comprises a plurality of rows of print nozzles;
the textile substrate is a compression or support device for the lower limbs or the upper limbs of the human body. It therefore becomes possible to print patterns or representations, possibly of complex shape, onto devices intended to treat a patient, in particular for lymphoedema, i.e. swelling of a part of the body following an accumulation of lymphatic fluid in the interstitial tissues;
the textile substrate is of the knitted, woven, lace or non-woven type;
the print nozzles are also capable of spraying a one-component silicone composition. Thus, it is possible to spray a one-component and two-component silicone composition with the same print nozzles, which reduces the production costs of textile printing and brings flexibility and convenience to the use of the printing system.

The invention also pertains to a printing system for inkjet printing a two-component silicone-based composition onto a textile substrate, said composition comprising a first component comprising at least one silicone polymer and a second component comprising at least one cross-linking agent, the printing system comprising at least one print head itself comprising at least one print nozzle, said print nozzle comprising two outlet orifices, including a first outlet orifice which is capable of spraying the first component, and a second outlet orifice which is capable of spraying the second component, and means for relative displacement between the print head and the textile substrate.

By virtue of the presence of two distinct outlet orifices for spraying the first component and the second component respectively onto the textile substrate, the silicone polymer is prevented from cross-linking in the print nozzle, or at least in the printing system, i.e. before being sprayed onto the textile substrate in the form of droplets. This makes it possible to avoid degradation of the printing system, or even of rendering the printing system inoperative and making printing impossible.

It should be noted that when the printing system comprises several print nozzles, one or more of them may comprise two outlet orifices as described above, and one or more of them may comprise a single outlet orifice.

Advantageously, the printing system may be used for inkjet printing a two-component composition, but also for inkjet printing a one-component composition, and in fact using the same print nozzles.

In accordance with other advantageous characterizing features of the invention, taken individually or in combination:
the printing system comprises a plurality of rows of print nozzles. Thus, the system of the invention can be used to improve the rate of production of the textile covered with silicone material by increasing the speed of travel of the textile without subjecting the print nozzles to excessively high stresses;
the rows of print nozzles form a rectangular matrix;
the print nozzles are also capable of spraying a one-component silicone composition. Thus, it is possible to spray a one-component and two-component silicone composition with the same print nozzles, which reduces the production costs of textile printing and brings flexibility and convenience to the use of the printing system. Furthermore, the presence of two outlet orifices for the same print nozzle increases the speed of printing the single-component silicone;
the print head is in the range 1 cm to 20 cm high, in the range 1 cm to 5 cm long and in the range 1 cm to 5 cm wide;
the rectangular matrix comprises at least 20 print heads in length and at least 10 print heads in width;
at least one print nozzle has a diameter in the range 0.5 cm to 3 cm, and comprises at least one outlet orifice having a diameter in the range 0.1 mm to 0.9 mm;
the displacement means comprise a mechanism for translation of the print head;
the displacement means comprise a mechanism for rotating or tilting the print head;
the displacement means comprise a mechanism for running the textile element in front of the print head;
the running mechanism comprises two rotating rollers and a support driven by the rollers and capable of receiving the textile substrate;
the running mechanism comprises two rotating rollers for respectively unwinding and winding the textile substrate;
the running mechanism comprises rods for guiding the textile substrate;
the running mechanism comprises a lateral guide plate for the textile substrate;
the running mechanism comprises a rotatable base and at least two rollers rotatably mounted on the base and each being capable of receiving a textile substrate;
the silicone composition comprises additives in the first component and/or in the second component;
the printing system comprises a device for controlling the quantity of silicone-based composition sprayed at each jet;
the control device controls the quantity of silicone composition sprayed individually by each nozzle. Thus, in operation, all of the nozzles can spray the silicone-based composition simultaneously, or only some of the nozzles can spray the silicone-based composition simultaneously;
the printing system includes a device for controlling the spacing between the nozzles.

The invention also pertains to the use of a printing system as described above for inkjet printing a one-component silicone-based composition and/or a two-component silicone-based composition.

### BRIEF DESCRIPTION OF THE DRAWINGS

The invention will be better understood from the following description which is given solely by way of non-limiting example and is made with reference to the accompanying drawings, in which:

FIG. 1 is a perspective view of a printing system in accordance with a first embodiment of the invention;

FIG. 2 is a top view along the arrow II of FIG. 1.

FIG. 3 is a side view along the arrow Ill of FIG. 1.

FIG. 4 is a front view along the arrow IV of FIG. 1.

FIG. 5 is a perspective view of a printing system in accordance with a second embodiment of the invention.

FIG. 6 is a top view along the arrow VI of FIG. 5.

FIG. 7 is a side view along the arrow VII of FIG. 5.

FIG. 8 is a front view along the arrow VIII of FIG. 5.

FIG. 9 is a perspective view of a printing system in accordance with a third embodiment of the invention;

FIG. 10 is a top view along the arrow X of FIG. 9.

FIG. 11 is a side view along the arrow XI of FIG. 9.

FIG. 12 is a diagrammatic representation of a textile article, of the support stocking type, comprising a textile element uniformly coated with flexible material at the upper edge.

FIG. 13 is a diagrammatic representation similar to FIG. 12, the textile element being coated in accordance with a pattern formed by a matrix of dots.

FIG. 14 is a diagrammatic representation similar to FIG. 12, the textile element being coated in accordance with a pattern formed by oblique bands.

FIG. 15 is a diagrammatic representation similar to FIG. 12, comprising several textile elements coated with flexible material at the upper edge, in the upper portion and in the central portion.

FIG. 16 is a diagrammatic representation similar to FIG. 15, the textile elements being coated in a different manner.

FIG. 17 is a diagrammatic representation of a print head array in accordance with one embodiment.

FIG. 18 is a diagrammatic perspective representation of a print head forming part of an array in accordance with FIG. 17.

FIG. 19 is a diagrammatic representation from below along arrow XII in FIG. 18.

FIG. 20 is a diagrammatic perspective representation of a print head comprising two print nozzles.

FIG. 21 is a diagrammatic representation of a print head comprising a print nozzle provided with two concentric outlet orifices.

FIG. 22 is a diagrammatic representation of a print head comprising a print nozzle provided with two outlet orifices which are separated from each other.

### DETAILED DESCRIPTION OF THE INVENTION

The subject matter of the invention is a method for inkjet printing a silicone-based composition onto a textile substrate. The silicone composition is two-component, i.e. it comprises two components intended to be mixed with each other when printing a pattern onto the textile substrate.

The method employs a printing system. For the purposes of simplification, the printing method and the printing system will be described at the same time in the remainder of the present text.

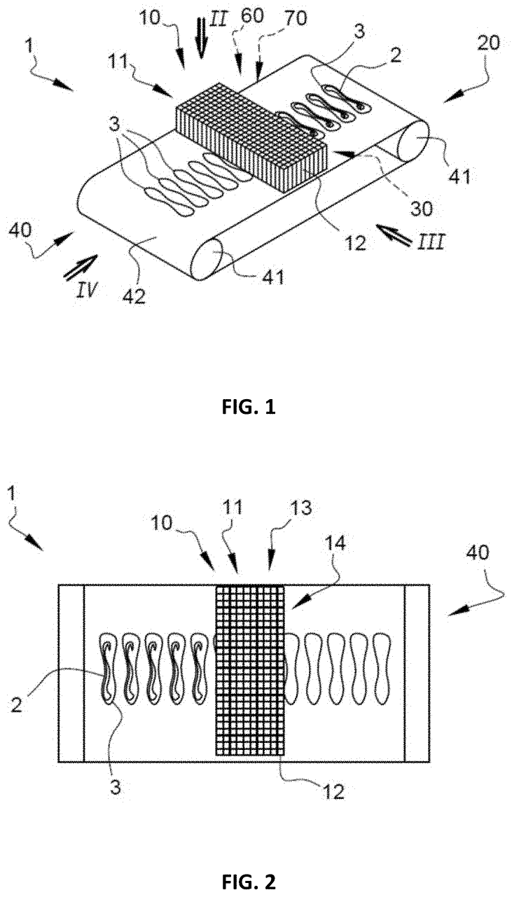

With reference to FIG. 1 which shows a printing system 1 in accordance with a first embodiment of the invention, the printing system 1 comprises at least one print head 10 comprising at least one print nozzle 12 which is capable of spraying the two-component silicone-based composition 2 onto the textile substrate 3, and means 20 for relative displacement between the print head 10 and the textile substrate.

The method comprises a step for spraying the first component and the second component onto the textile substrate 3 via the print nozzles 12 of the printing system 1.

The term “silicone-based composition” is used to mean a composition comprising one or more silicone polymers. A silicone polymer, also known as a silicone elastomer, is composed of silicon-oxygen Si—O chains comprising chemical functions bonded to silicon atoms.

When the silicone polymer is sprayed from the print nozzle toward the textile substrate, said polymer cross-links to form a printed pattern on said textile substrate. Cross-linking corresponds to the formation of one or more three-dimensional networks, by chemical or physical means. During cross-linking, chemical bonds, known as bridges, are created between the macromolecular chains of silicone polymer. Cross-linking may occur at the outlet from the print nozzle, before the composition comes into contact with the textile substrate, or when the composition comes into contact with the textile substrate. Cross-linking may occur by polyaddition or by polycondensation.

As indicated above, the silicone-based composition is a two-component composition. A first component comprises at least one silicone polymer, and a second component comprises a cross-linking agent.

The cross-linking agent is capable of initiating cross-linking of the silicone polymer when it comes into contact therewith, i.e. when the first component and the second component are mixed. Mixing of the two components may occur before the components come into contact with the textile substrate, or when the components contact the textile substrate. Preferably, the silicone-based composition comprises at least 50% by weight of silicone polymer with respect to the total weight of the composition, and more preferably at least 70%. The weight of the composition corresponds to the sum of the weight of the first component and the weight of the second component.

The silicone polymer is advantageously compatible with skin, i.e. it is suitable for contact. This means that the silicone polymer does not present any biological or toxicological risk when in contact with the skin and does not cause a skin reaction.

In practice, the thickness of the silicone layer is preferably in the range 0.1 mm to 2 mm.

The textile substrate is preferably a device or a compression and/or support article for the lower limbs or the upper limbs of the human body. The method of the invention thus makes it possible to print patterns or representations, optionally of complex shape, onto devices or articles intended to treat a patient, in particular for lymphoedema.

The textile substrate may, for example, comprise footwear (socks, hold-up stockings, for example) and non-footwear of the adjustable compression or support article type (elastic and inelastic), as well as armsleeve, or gloves.

The textile substrate may in particular be of the knitted, woven, lace or non-woven type.

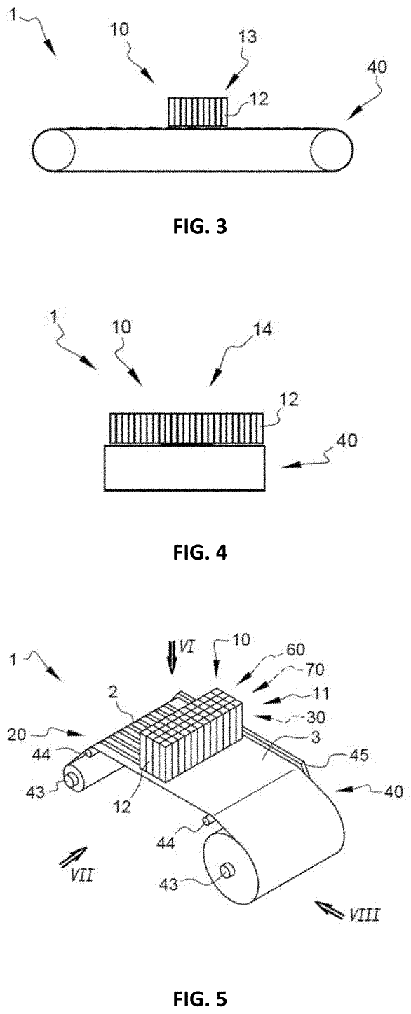

FIGS. 1 to 4 show a printing system 1 in accordance with a first embodiment of the invention.

The system 1 comprises one or more print heads 10 and means 20 for relative displacement between the print head(s) 10 and the textile substrate 3. A plurality of print heads 10 may be designated as a print head array.

Each print head 10 comprises at least one print nozzle 12, making it possible to spray the silicone-based composition onto a specific zone 4 of the textile substrate 3.

Preferably, the printing system 1 is suitable for printing both a two-component composition and a one-component composition.

In the case of inkjet printing a one-component silicone-based composition, the polymer and the cross-linking agent are contained in the same component. The polymer may be cold-cross-linkable or hot-cross-linkable.

For a cold-cross-linkable single-component silicone polymer, cross-linking is initiated by the moisture in the air, i.e. when the composition is expelled from the print nozzle and comes into contact with the ambient air. For a single-component silicone polymer which can be cross-linked when hot, the composition is first sprayed onto the textile substrate 3, then the temperature is increased until cross-linking is initiated, preferably to a temperature in the range 90° C. to 200° C. at atmospheric pressure. The cross-linking temperature obviously depends on the nature of the silicone polymer.

Preferably, each print head 10 comprises a plurality of print nozzles 12. The print nozzles are preferably disposed in a plurality of rows of nozzles 12. A row of print nozzles 12 comprises a minimum of two print nozzles.

Preferably, the rows 11 of print nozzles 12 form a rectangular matrix 13, 14, consisting of mutually parallel rows 13 and mutually parallel columns 14 which are perpendicular to the rows 13.

In accordance with an optional variation, the print head 10 may comprise several rows 11 of print nozzles 12 which are parallel to one another but offset with respect to one another, such that the nozzles 12 are disposed in a honeycomb arrangement.

In accordance with one embodiment, the printing system 1 comprises at least one first print nozzle 12a which is capable of spraying the first component, and at least one second print nozzle 12b which is capable of spraying the second component of the two-component silicone-based composition onto the textile substrate 3.

Preferably, the first print nozzle 12a and the second print nozzle 12b belong to the same print head 10, as illustrated in FIG. 20. With reference to FIG. 20, the print nozzle 12a is capable of spraying the first component, and the second print nozzle 12b is capable of spraying the second component of the two-component silicone-based composition onto the textile substrate 3.

This is advantageous when the printing system is used for printing a one-component silicone composition, since the rate of production is improved. When the two-component silicone composition is used, in addition to an improvement in production, the silicone polymer is prevented from cross-linking in the print nozzles, or at least in the printing system, i.e. before arriving on the textile substrate. This makes it possible to prevent degradation of the printing system, or even of rendering the printing system inoperative and making printing impossible.

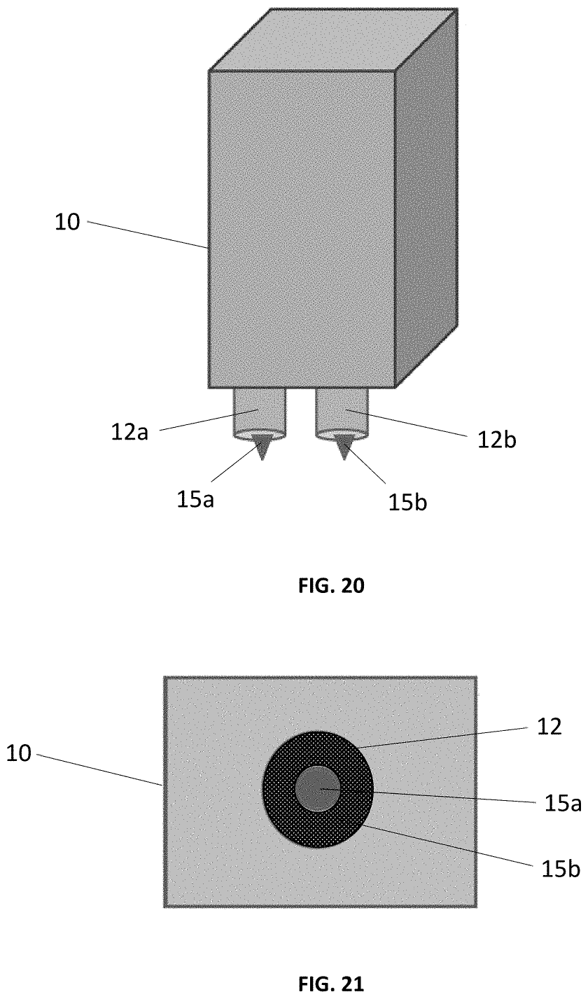

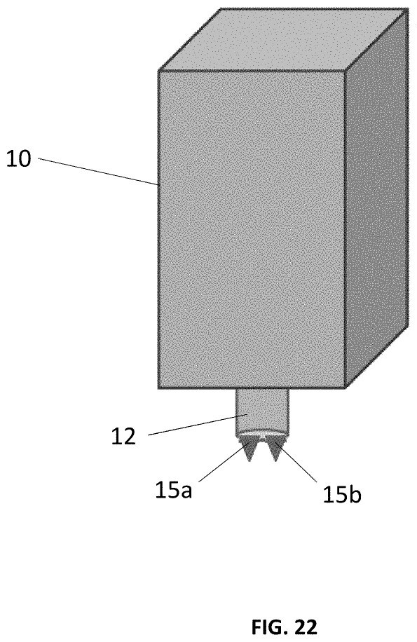

In accordance with an embodiment shown in FIGS. 21 and 22, the printing system 1 comprises at least one print nozzle 12 comprising two outlet orifices, wherein a first outlet orifice 15a is capable of spraying the first component, and a second outlet orifice 15b is capable of spraying the second component.

The first outlet orifice 15a and the second outlet orifice 15b may be concentric, as shown in FIG. 21, or they may be separate from each other, as shown in FIG. 22.

As regards the displacement means 20, these may include a mechanism 30 for translation of the print head 10.

In accordance with a preferred embodiment, the translation mechanism 30 is configured to carry out transverse and/or longitudinal translation of the print head 10 allowing, for example, flexible material 2 to be sprayed in the form of oblique bands.

In accordance with an optional variation, the displacement means 20 may comprise a mechanism for rotating or tilting the print head 10.

In accordance with another variation, the print head 10 may be fixed within the system 1.

Furthermore, the displacement means 20 comprise a mechanism 40 for moving the textile substrate 3 beneath the print head 10, in a translational and/or rotational movement.

The running mechanism 40 comprises two rotating rollers 41 and a support 42 driven by the rollers 41 and designed to receive the textile substrate 3.

In the example of FIGS. 1 to 4, the running mechanism 40 is a smooth belt conveyor consisting of rollers 41 rotating about their respective axes which are parallel to each other. The smooth belt of the conveyor is a flexible support 42 wrapped around the rollers 41.

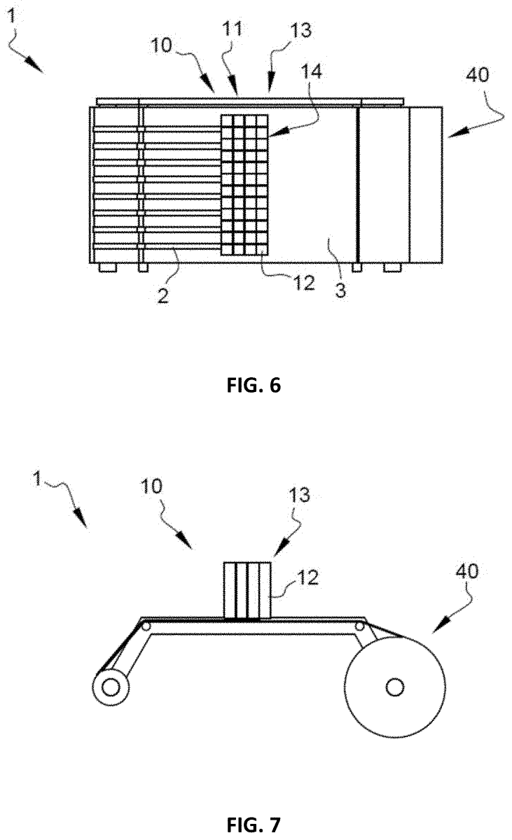

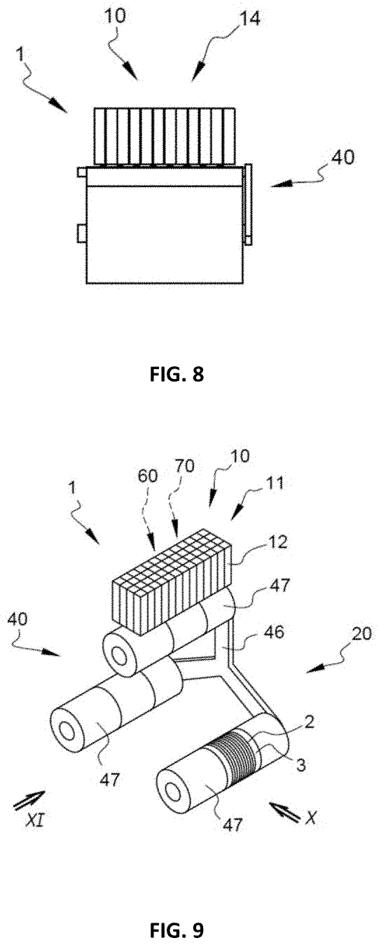

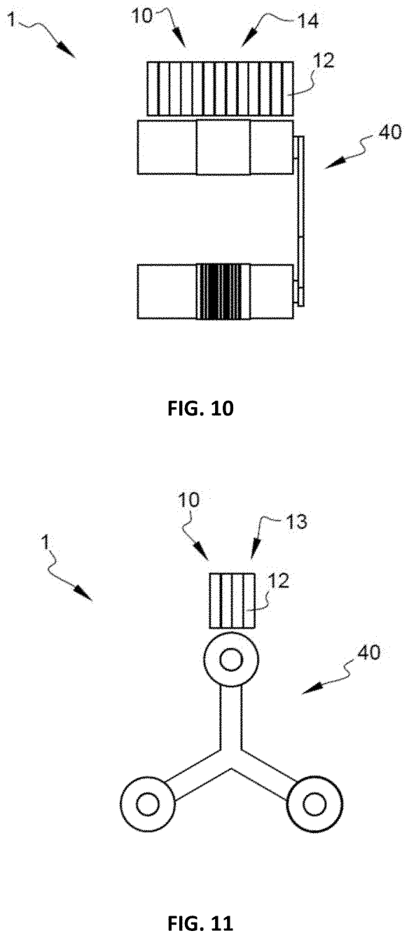

Other running mechanisms 40 may of course be envisaged without departing from the scope of the invention, as illustrated in FIGS. 5 to 11.

Advantageously, the system 1 may comprise a device 60 for controlling the quantity of flexible material 2 sprayed at each jet. The piloting device 60 may be configured to control the quantity of silicone-based composition 2 individually sprayed from each nozzle.

Moreover, in operation, all of the print nozzles 12 can spray the silicone composition simultaneously, or in fact only certain nozzles 12 can spray the silicone composition simultaneously.

The use of this control device 60 therefore allows for uniform coverage of an extensive zone 4 of the textile substrate 3 produced from flexible material 2 if the device 60 is configured so that all of the nozzles 12 spray flexible material 2 simultaneously.

Uniform coverage is also possible thanks to this control device 60, by controlling the quantity of silicone-based composition 2 sprayed by the print nozzles 12 even if only certain nozzles 12 spray said composition 2.

On the other hand, if the device 60 allows only certain nozzles 12 to spray silicone-based composition, partial coverage of the textile substrate 3 by said composition is possible, in particular in the form of patterns.

Optionally, the system 1 may include a device 70 for controlling the spacing between the print nozzles 12. This control device 70 makes it possible, inter alia, to increase the coated zone of the textile substrate 3 or alternatively to print the textile substrate 3 by spraying a matrix of dots of silicone-based composition.

In practice, a matrix of dots of silicone-based composition 2, as illustrated in FIGS. 13, 15 and 16, can be sprayed either by means of the control device 60, by reducing the quantity of composition 2 sprayed by the matrix of nozzles 12, or by means of the control device 70, by uniformly spacing the nozzles 12 with respect to one another.

Other embodiments of a printing system 1 in accordance with the invention are shown in FIGS. 5 to 11. Certain constituent elements of the system 1 are comparable to those of the first embodiment described above and have the same numerical references for the purposes of simplification.

FIGS. 5 to 8 show a printing system 1 in accordance with a second embodiment of the invention.

The running mechanism 40 comprises two rotating rollers 43 provided respectively for unwinding and winding the textile substrate 3, two longitudinal guide rods 44 and a lateral guide plate 45.

More precisely, the two rollers 43 comprise an upstream roller 43 for unwinding and a downstream roller 43 for winding the textile substrate 3 in the form of a wide textile web.

This textile web runs in a flat manner under the print head 10, between the guide rods 44.

Thus, the guide rods 44 and the lateral guide plate 45 make it possible to guide the textile web longitudinally and laterally, from the upstream roller 43 to the downstream roller 43, while receiving a coating of flexible material 2 when the textile web travels between the rods 44.

FIGS. 9 to 11 show a printing system 1 in accordance with a third embodiment of the invention.

The running mechanism 40 comprises a rotating base 46 and at least two rollers 47 rotatably mounted on the base 46 and each designed to receive a textile substrate 3.

The base 46 is movable in rotation about its central axis. The base 46 comprises three branches 48 and three rods 49 fixed to the ends of the branches 48. The branches 48 extend radially with respect to the central axis, while the rods 49 extend parallel to the central axis. The rollers 47 are hollow and rotatably mounted on the rods 49.

Alternatively, the base 46 may have any shape which is suitable for supporting a plurality of rollers 47, for example two or four rollers 47 rotatably mounted on the rods 49 of the base 46.

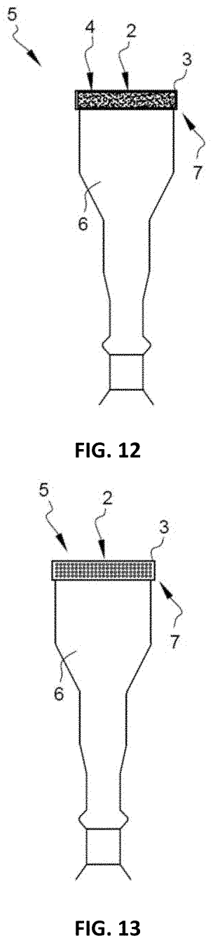

FIGS. 12 to 16 show different embodiments of textile articles 5 produced by implementing the system 1 in accordance with the invention.

The textile articles 5 are of the support stocking type 6, comprising an upper edge 7 provided with an opening for insertion of the leg.

Each upper edge is provided with a textile substrate 3 on which a pattern is printed, the material of which being based on a cross-linked silicone polymer. For the purposes of simplification, the pattern produced from a material based on a cross-linked silicone polymer bears the same legend “2” as the silicone-based composition from which it is derived.

In accordance with a first embodiment, the textile substrate 3 is in the form of a strip attached to the stocking 6. In a second embodiment, the textile substrate 3 is constituted directly by the top edge 7 of the stocking 6, covered with the pattern 2 by virtue of the printing system 1.

In FIG. 12, the pattern 2 uniformly covers a specified extensive zone 4 of the textile substrate 3.

In FIG. 13, the pattern 2 partially covers the textile substrate 3 in the form of a matrix of dots.

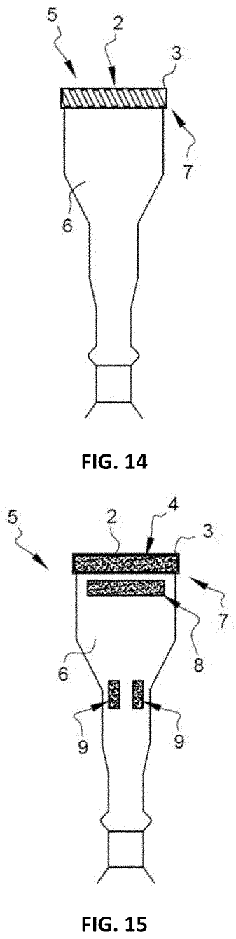

In FIG. 14, the pattern 2 partially covers the textile substrate 3 in the form of parallel oblique bands.

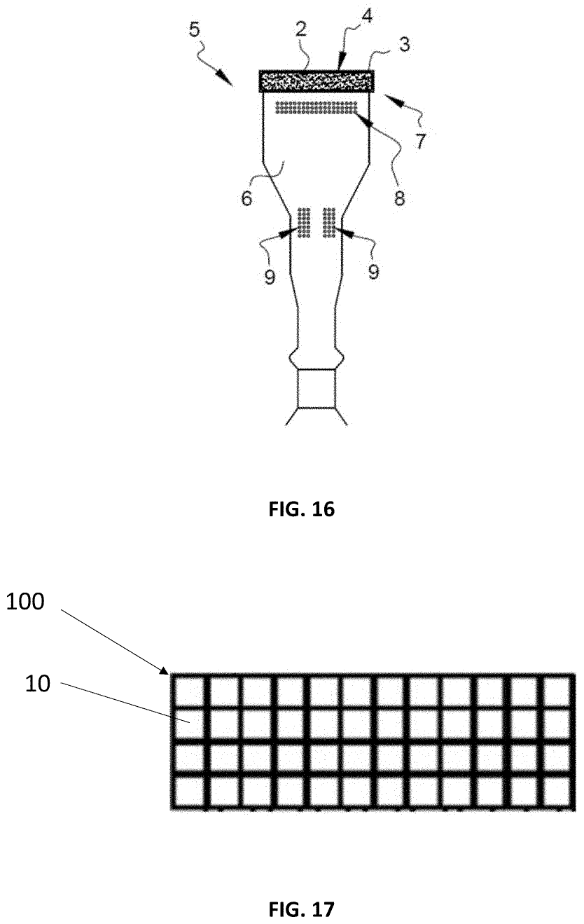

In FIGS. 15 and 16, the textile article 5 has a pattern 2 at the top edge 7, at the top portion 8 close to the edge 7, and at the central portion 9 of the support stocking 6. These different zones 8, 9 comprise a printed pattern 2, the printing being uniform as in FIG. 15, or partial in the form of a matrix of dots as in FIG. 16. These different zones 8, 9 receive the pattern 2 either directly by printing, or via an added textile substrate 3.

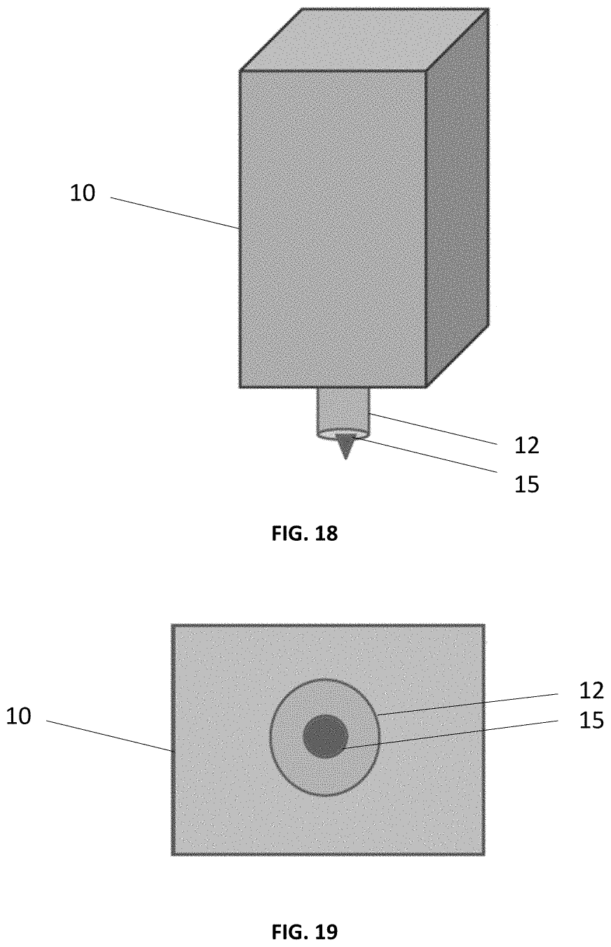

In accordance with a preferred embodiment illustrated in FIGS. 17 to 19, the printing system 1 comprises a plurality of print heads 10 combined in the form of one or more sets, termed one or more arrays 100 of print heads. The print heads 10 are arranged so as to form one or more rows as well as several columns. The array 100 of print heads 10 advantageously forms a rectangular matrix.

Preferably, an array 100 of print heads 10 forms a rectangular matrix comprising approximately twenty print heads in length and approximately ten print heads in width. The print heads 10 each preferably have a height of 10 cm, a width of 5 cm and a length of 5 cm, thus forming an array of 200 print heads having a length of 1 metre and a width of 50 cm.

In practice, the print heads 10 have a height which is preferably in the range 1 cm to 20 cm, a length which is preferably in the range 1 cm to 5 cm, and a width which is preferably in the range 1 cm to 5 cm. Each print head 10 comprises at least one print nozzle 12 which is preferably cylindrical in shape and provided with an outlet orifice 15. The print nozzle preferably has a diameter in the range 0.5 cm to 3 cm, and the outlet orifice 15 preferably has a diameter in the range 0.1 mm to 0.9 mm.

The printing system 1 may also comprise a device for controlling the quantity of silicone-based composition sprayed by the print heads 10 of each array 100, or in fact a device for controlling the spacing between the print heads 10 of an array 100.

Moreover, the printing system 1 and articles 5 may be configured differently from FIGS. 1 to 21 without departing from the scope of the invention as defined in the claims. In particular, the material based on a cross-linked silicone polymer 2 may be printed in any zones and in any patterns, depending on the intended application. Furthermore, the technical features of the various embodiments and variations mentioned above may be combined in their entirety or only in part. Thus, the system 1 can be adapted in terms of cost, functions and performance.

1) A method for ink-jet printing a two-component silicone-based composition onto a textile substrate, said composition comprising a first component comprising at least one silicone polymer and a second component comprising at least one cross-linking agent, the method comprising the steps of:
providing a printing system comprising at least one print head itself comprising at least one print nozzle which is capable of spraying the two-component silicone-based composition onto the textile substrate, and a device for producing relative displacement between the print head and the textile substrate,
spraying the first component and the second component onto the textile substrate via said print nozzle, and
cross-linking the silicone polymer by reaction between the first component and the second component in order to form a pattern printed on the textile substrate.

2) The printing method as claimed in claim 1, characterized in that cross-linking of the silicone polymer is initiated by mixing the first component and the second component on the textile substrate or before coming into contact with the textile substrate.

3) The printing method as claimed in claim 1, characterized in that the two-component silicone-based composition comprises at least 50% by weight of silicone polymer with respect to the weight of said composition.

4) The printing method as claimed in claim 1, characterized in that the two-component silicone-based composition comes into contact with the skin.

5) The printing method as claimed in claim 1, characterized in that the printing system comprises at least one first print nozzle which is capable of spraying the first component, and at least one second print nozzle which is capable of spraying the second component.

6) The printing method as claimed in claim 5, characterized in that the first print nozzle and the second print nozzle belong to the same print head.

7) The printing method as claimed in claim 5, characterized in that the first print nozzle and the second print nozzle belong to two distinct print heads.

8) The printing method as claimed in claim 1, characterized in that at least one print nozzle comprises two outlet orifices, including a first outlet orifice which is capable of spraying the first component, and a second outlet orifice which is capable of spraying the second component.

9) The printing method as claimed in claim 1, characterized in that the textile substrate is a compression or support device for the lower limbs or the upper limbs of the human body.

10) The printing method as claimed in claim 1, characterized in that the textile substrate is of the knitted, woven, lace or non-woven type.

11) A printing system for ink-jet printing a two-component silicone-based composition onto a textile substrate, said composition comprising a first component comprising at least one silicone polymer and a second component comprising at least one cross-linking agent, the printing system comprising at least one print head itself comprising at least one print nozzle said print nozzle comprising two outlet orifices, including a first outlet orifice which is capable of spraying the first component, and a second outlet orifice which is capable of spraying the second component, and a device for producing relative displacement between the print head and the textile substrate.

12) The printing system as claimed in claim 11, characterized in that the system comprises several rows of print nozzles forming a rectangular matrix.

13) The printing system as claimed in claim 12, characterized in that the rectangular matrix comprises at least twenty print heads in length and at least ten print heads in width.

14) The printing system as claimed in claim 11, characterized in that at least one print head has a height in the range 1 cm to 20 cm, a length in the range 1 cm to 5 cm, and a width in the range 1 cm to 5 cm.

15) The printing system as claimed in claim 11, characterized in that at least one print nozzle has a diameter in the range 0.5 cm to 3 cm and comprises at least one outlet orifice having a diameter in the range 0.1 mm to 0.9 mm.

16) Use of a printing system as claimed in claim 11 for ink-jet printing a one-component silicone-based composition and/or a two-component silicone-based composition.
